(19) United States
(12) Patent Application Publication
(10) Pub. No.: US 2017/0056668 A1
(43) Pub. Date: Mar. 2, 2017

(54) CARDIAC PACING WITH GOVERNOR

(71) Applicant: Cardiac Pacemakers, Inc., St. Paul, MN (US)

(72) Inventors: Keith R. Maile, New Brighton, MN (US); Michael J. Kane, Roseville, MN (US); William J. Linder, Golden Valley, MN (US)

(73) Assignee: Cardiac Pacemakers, Inc., St. Paul, MN (US)

(21) Appl. No.: 15/244,694

(22) Filed: Aug. 23, 2016

Related U.S. Application Data
(60) Provisional application No. 62/209,136, filed on Aug. 24, 2015.

(51) Int. Cl. A61N 1/365; A61B 5/042; A61B 5/0452; A61N 1/375

(52) U.S. Cl. CPC A61N 1/36585; A61N 1/3756; A61B 5/0422; A61N 1/36514; A61N 1/36542; A61B 5/0452

(57) ABSTRACT

A leadless cardiac pacemaker includes a housing and two or more electrodes secured relative to the housing. A controller is configured to receive electrical signals from the two or more electrodes indicative of a heartbeat of the patient's heart and determine a first measure of cardiac load based at least in part on a delay between the Q or R feature and the T feature for the heartbeat. An accelerometer is configured to sense an activity level of the patient and the controller is configured to determine a second measure of cardiac load based at least in part on the activity level of the patient. A pacing rate is based at least in part upon the first measure of cardiac load, with the second measure of cardiac load used as a governor to the determined pacing rate.

### CROSS REFERENCE TO RELATED APPLICATIONS

This application claims the benefit of U.S. Provisional Patent Application Ser. No. 62/209,136 filed on Aug. 24, 2015, the disclosure of which is incorporated herein by reference.

### TECHNICAL FIELD

The present disclosure generally relates to systems, devices, and methods for pacing the heart of a patient, and more particularly, to systems, devices, and methods for rate responsive pacing of the heart of a patient.

### BACKGROUND

Pacing instruments can be used to treat patients suffering from various heart conditions that result in a reduced ability of the heart to deliver sufficient amounts of blood to a patient's body. These heart conditions may lead to rapid, irregular, and/or inefficient heart contractions. To help alleviate some of these conditions, various devices (e.g., pacemakers, defibrillators, etc.) can be implanted into a patient's body. Such devices may monitor and provide electrical stimulation to the heart to help the heart operate in a more normal, efficient and/or safe manner. In some cases, such devices may pace the patient's heart at a pacing rate that adapts to changes in a patients' physical activity. This is often referred to as rate-responsive pacing, and may help match the cardiac output of the patient's heart with the cardiac load caused by the patient's current activity level.

### SUMMARY

The present disclosure generally relates to systems, devices, and methods for pacing a patient's heart in accordance with a first measure of cardiac load and more particularly to using a second measure of cardiac load as a governor to the pacing rate determined by the first measure of cardiac load. While not so limited, in a first illustrative embodiment, a leadless cardiac pacemaker (LCP) that is configured to sense and pace a patient's heart may include a housing, two or more electrodes secured relative to the housing, and a controller. The two or more electrodes may be configured to receive electrical signals emanating from the patient's heart. The controller may be coupled to the two or more electrodes, and may receive electrical signals from the two or more electrodes indicative of a heartbeat of the patient's heart. The received electrical signals may include one or more of a Q, an R, and an S feature, as well as a T feature of a QRST indicative of a heartbeat. The controller may be configured to determine a first measure of cardiac load based at least in part on a delay between the Q or R feature and the T feature of the heartbeat. While not so limited, in some cases, the controller determines the first measure of cardiac load based at least in part on a delay between the R feature and the T feature of the heartbeat.

The illustrative LCP may further include an accelerometer or other sensor that is configured to sense an activity level of the patient. The controller may then be configured to determine a second measure of cardiac load based at least in part on the sensed activity level of the patient. The controller may determine a pacing rate based at least in part on the first measure of cardiac load and to pace the patient's heart in accordance with the determined pacing rate. The controller may further be configured to utilize the second measure of cardiac load to act as a governor to the determined pacing rate.

Alternatively or additionally to any of the variations described above with respect to the first illustrative embodiment, the controller may be configured to receive electrical signals from the two or more electrodes of the LCP indicative of a plurality of heartbeats, and to determine the first measure of cardiac load based at least in part on a delay between the Q or R feature and the T feature of two or more heartbeats.

Alternatively or additionally to any of the variations described above with respect to the first illustrative embodiment, the controller may be configured to repeatedly activate and deactivate the accelerometer to intermittently sense the activity level of the patient, and determine the second measure of cardiac load based at least in part on one, two or more intermittently sensed activity levels of the patient.

Alternatively or additionally to any of the variations described above with respect to the first illustrative embodiment, the controller may be configured to act as a governor by determining a pacing rate limit based at least in part on the second measure of cardiac load, and to limit the determined pacing rate based on the pacing rate limit. In some cases, the pacing rate limit may be a function of the second measure of cardiac load, and the function may not be a constant function.

Alternatively or additionally to any of the variations described above with respect to the first illustrative embodiment, the controller may be configured to determine the pacing rate based at least in part upon the first measure of cardiac load using a linear function having a non-zero slope.

Alternatively or additionally to any of the variations described above with respect to the first illustrative embodiment, the accelerometer may be configured to sense non-cardiac movement of the patient, and the activity level of the patient may be based on the sensed non-cardiac movement of the patient.

Alternatively or additionally to any of the variations described above with respect to the first illustrative embodiment, the accelerometer may be configured to sense cardiac movement, and the activity level of the patient may be based on the sensed cardiac movement.

Alternatively or additionally to any of the variations described above with respect to the first illustrative embodiment, the accelerometer may be configured to sense non-cardiac movement of the patient and cardiac movement, and the activity level of the patient may be based at least in part on the sensed non-cardiac movement of the patient and the sensed cardiac movement.

In a second illustrative embodiment, a leadless cardiac pacemaker (LCP) configured to sense and pace a patient's heart may include a housing, two or more electrodes secured relative to the housing, and a controller. The two or more electrodes may be configured to receive electrical signals emanating from the patient's heart. The controller may be coupled to the two or more electrodes, and may receive electrical signals from the two or more electrodes and determine a first measure of cardiac load based on the received electrical signals. The LCP may further include a sensor that provides a second measure of cardiac load to the controller. The controller may determine a pacing rate based at least in part upon the first measure of cardiac load and to pace the patient's heart in accordance with the determined pacing rate. The controller may further use the second measure of cardiac load to determine a pacing rate limit, and to limit the determined pacing rate based on the pacing rate limit.

Alternatively or additionally to the second illustrative embodiment described above, the first measure of cardiac load may be representative of how quickly the patient's heart prepares for a subsequent heartbeat.

Alternatively or additionally to any of the variations described above with respect to the second illustrative embodiment, the first measure of cardiac load may be representative of a time required for ventricle depolarization and repolarization.

Alternatively or additionally to any of the variations described above with respect to the second illustrative embodiment, the first measure of cardiac load may comprise an RT interval.

Alternatively or additionally to any of the variations described above with respect to the second illustrative embodiment, the second measure of cardiac load may be representative of gross movement of the patient.

Alternatively or additionally to any of the variations described above with respect to the second illustrative embodiment, the sensor may comprise an accelerometer and the second measure of cardiac load may be derived at least in part from a signal outputted from the accelerometer.

Alternatively or additionally to any of the variations described above with respect to the second illustrative embodiment, the controller may be further configured to temporarily ignore any changes in the first measure of cardiac load, for at least two heartbeats, in determining the pacing rate.

Alternatively or additionally to any of the variations described above with respect to the second illustrative embodiment, the controller may be further configured to determine a plurality of first measures of cardiac load over time based on the received electrical signals, and to determine the pacing rate based on a moving average of two or more of the plurality of first measures of cardiac load.

In a third illustrative embodiment, a leadless cardiac pacemaker (LCP) configured to sense and pace a patient's heart may include two or more electrodes configured to receive electrical signals emanating from the patient's heart, and a controller coupled to the two or more electrodes. The controller may be configured to ascertain a first measure of cardiac load based at least in part upon electrical signals received by the two or more electrodes. The controller may further be configured to receive a second measure of cardiac load, and to determine a pacing rate based at least in part upon the first measure of cardiac load and the second measure of cardiac load. The controller may pace the patient's heart in accordance with the determined pacing rate. In some cases, the first measure of cardiac load may include a measure that responds more quickly to changes in cardiac activity than the second measure of cardiac load.

The above summary is not intended to describe each embodiment or every implementation of the present disclosure. Advantages and attainments, together with a more complete understanding of the disclosure, will become apparent and appreciated by referring to the following description and claims taken in conjunction with the accompanying drawings.

### BRIEF DESCRIPTION OF THE DRAWINGS

The disclosure may be more completely understood in consideration of the following description of various illustrative embodiments in connection with the accompanying drawings, in which:

FIG. 1 is a schematic representation of an electrocardiograph (ECG) trace, identifying particular elements of the ECG trace;

FIG. 2 is a graphical representation illustrating a relationship between the QT interval and heart rate;

FIG. 3 is a schematic representation of ECG traces of several heart beats, illustrating a change in the QT interval and corresponding change in heart rate;

FIG. 4 is a schematic block diagram of an illustrative pacing control system;

FIG. 5 is a schematic block diagram of another illustrative pacing control system;

FIG. 6 is a schematic block diagram of another illustrative pacing control system;

FIG. 7 is a schematic block diagram of yet another illustrative pacing control system;

FIG. 8 is a graphical representation illustrating a relative weighting that can be applied between first and second indications of cardiac load, and the temporal relationship therebetween according to an embodiment of the disclosure;

FIG. 9 is a graphical representation illustrating a relationship between QT drive and governor response time according to an embodiment of the disclosure;

FIG. 10 is a schematic block diagram of an illustrative LCP according to an embodiment of the disclosure;

FIG. 11 is a schematic block diagram of another illustrative LCP;

FIG. 12 is a schematic block diagram of another illustrative LCP;

FIG. 13 is a schematic block diagram of yet another illustrative LCP;

FIG. 14 is a schematic block diagram of an illustrative medical device in accordance with the disclosure;

FIG. 15 is a schematic diagram of an illustrative medical system that includes multiple LCPs and/or other devices in communication with one another, in accordance with the disclosure; and

FIG. 16 is a schematic diagram of an illustrative medical system that includes an LCP and another medical device, in accordance with the disclosure.

While the disclosure is amenable to various modifications and alternative forms, specifics thereof have been shown by way of embodiment in the drawings and will be described in detail. It should be understood, however, that the intention is not to limit aspects of the disclosure to the particular illustrative embodiments described. On the contrary, the intention is to cover all modifications, equivalents, and alternatives falling within the spirit and scope of the disclosure.

### DESCRIPTION

The following description should be read with reference to the drawings in which similar elements in different drawings are numbered the same. The description and the drawings, which are not necessarily to scale, depict illustrative embodiments and are not intended to limit the scope of the disclosure.

A variety of different devices, including but not limited to leadless cardiac pacemakers (LCP), may be implanted on a patient or within a chamber of the patient's heart and may operate to sense physiological signals and parameters and deliver one or more types of electrical stimulation therapy to the heart of the patient. Example electrical stimulation therapy may include bradycardia pacing, rate responsive pacing therapy, cardiac resynchronization therapy (CRT), anti-tachycardia pacing (ATP) therapy and/or the like. In various pacing therapies such as those listed herein, one of the challenges is determining an optimal or otherwise desired pacing rate, particularly since the cardiac load depends on the patient's activity level. One of the particular potential challenges in determining an optimal pacing rate pertains to how the heart responds to rapid changes in cardiac load such as may occur when a patient transitions from a relatively sedentary activity to a more active activity. This may, for example, represent a cardiac patient standing up, or getting out of bed, or climbing a set of stairs, or any other activity. In this, the phrase “cardiac load” may be considered as representing or defining a measure of how hard the heart needs to beat to meet the blood flow requirements, or perhaps oxygen requirements, of the patient's body. There is a desire to adjust the pacing rate in accordance with the cardiac load. Generally, as the cardiac load increases, the pacing rate should also increase. As the cardiac load subsequently decreases, the pacing rate should also decrease and should eventually return to a baseline pacing rate that may be determined for a particular patient in a sedentary state. Adjusting the heart rate in such a manner is often referred to as rate responsive pacing.

An increase in cardiac load may be short term, or longer term. If a patient stands up to walk across a room and then sit down again, the increase in cardiac load may be measured in seconds. Once the patient sits down again, the cardiac load will drop quickly beyond perhaps a recovery period. A walk around the block may represent an increase in cardiac load measured in minutes. In determining an optimal pacing rate, it is useful to pay attention both to how cardiac load changes over a short period of time, but also longer term how the cardiac load returns closer to a baseline cardiac load. It will be appreciated that there are a number of ways that an implanted medical device such as an LCP may recognize changes in cardiac load. Electrical signals representing an ECG or portions thereof may provide indications of cardiac load and/or cardiac load changes. Respiration rate changes and blood temperature are also useful indications, as are gross body motions, posture and heart sounds.

With respect to electrical signals, contractility or peak endocardial acceleration can be used in determining a measure of cardiac load. Peak endocardial acceleration is an indication of how strongly the heart is pumping (how intensely the ventricle contracts). If the heart starts pumping more strongly, that can be a strong indication of an increase in cardiac load. Detecting peak endocardial acceleration can be sensed using an accelerometer, which is considered to be a relatively power hungry sensor. However, the accelerometer can be periodically turned on for a brief period of time, long enough to take a measurement, then power down again in order to conserve power. Another electrical signal that may be used in determining cardiac load is the QT interval. Blood temperature can also be used to determine cardiac load. However, blood temperature typically initially drops for some time before increasing with increased cardiac load. Because of this characteristic as well as a relatively long time constant, blood temperature is often not a good choice to detect short term changes in cardiac load.

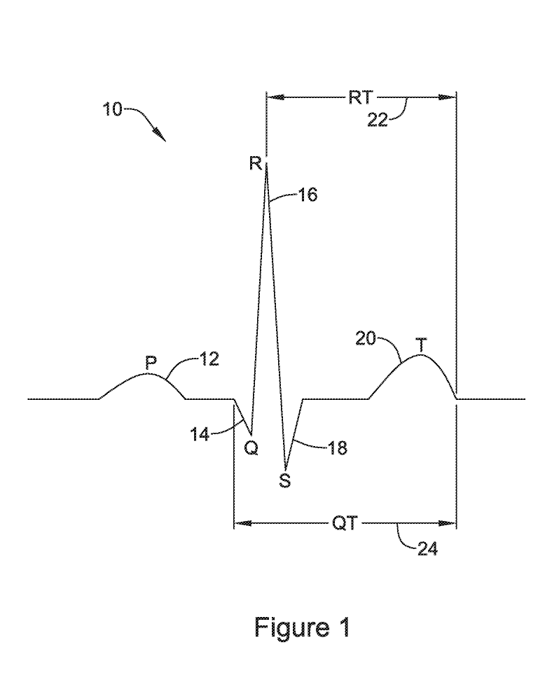

FIG. 1 shows an illustrative ECG trace 10 for a single heartbeat. In some cases, the ECG trace 10 may be referred to as a QRS signal, or a PQRS or even a PQRST signal, as the ECG trace 10 includes a P peak 12, a Q trough 14, an R peak 16, an S trough 18 and a T peak 20. An RT interval 22 is defined as the time between the R peak 16 and the T peak 20. A QT interval 24 is defined between the Q trough 14 and the T peak 20. In some cases, the QT interval 24 may be considered as representing a period of time necessary for the heart to prepare for a subsequent beat, including the time necessary for ventricular depolarization and repolarization. The RT interval may be easier to detect, since the R peak 16 is generally more intense (e.g. has a higher amplitude) than the Q trough 14. The RT interval 22 may be considered as being related to the QT interval 24, and may also represent the heart requires to prepare for a subsequent beat.

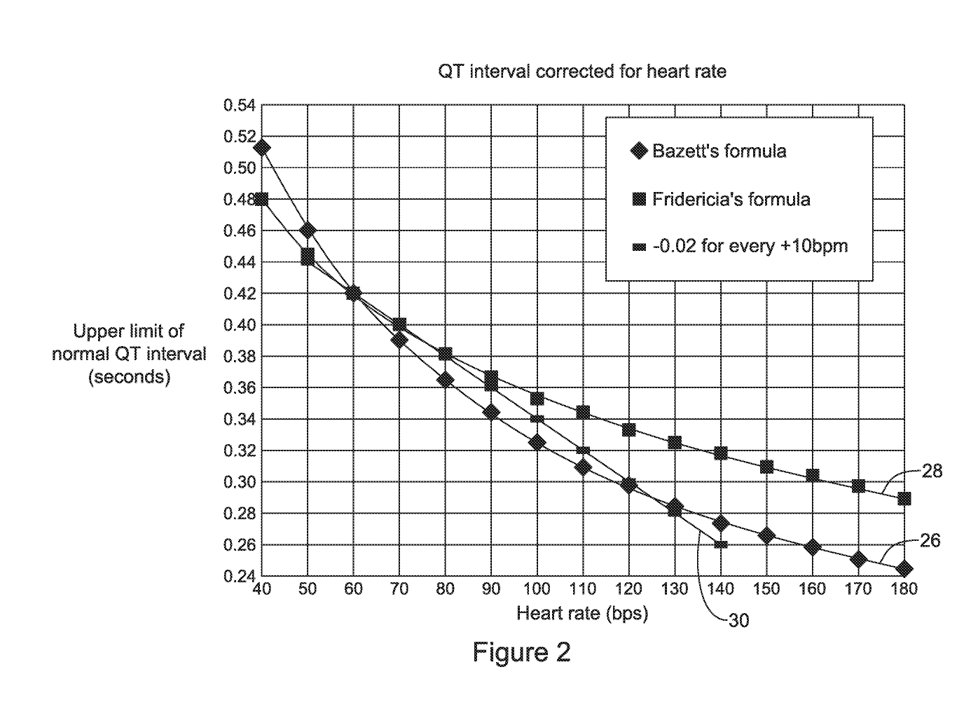

Generally, the QT interval is inversely proportional to the heart rate. As the heart rate increases, the QT interval shortens. Typically, the patient's body automatically shortens the QT interval with increased cardiac load. Thus, the QT interval can be used as an indication of current cardiac load, and can even be used to identify a desired heart rate. There are known relationships defined between the QT (or RT) interval and heart rate. FIG. 2 shows a graphical representation of several of these relationships. One relationship is the Bazett's formula, labeled as line 26 in FIG. 2. According to Bazett's formula, given a QT interval, the desired or corrected heart rate (HRC) is given by the formula:

$$HRC = \frac{QT}{\sqrt{RR}} ,$$

where RR is the interval from the onset of one QRS interval to the onset of the next QRS interval. Another relationship is Friderecia's formula, labeled as line 28 in FIG. 2. According to Friderecia's formula, the corrected heart rate is given by the formula:

$$HRC = \frac{QT}{\sqrt[3]{RR}} .$$

Yet another formula shown in FIG. 2, labeled as line 30, subtracts 0.2 seconds from the QT interval for every 10 beats per minute (bpm) increase in heart rate. While not illustrated in FIG. 2, additional relationships include the Framingham formula and Hodge's formula, given by the following formulas, respectively:

HRC=QT+0.154(1−RR)

HRC=QT+1.75(heart rate−60).

It will be appreciated that other relationships may also be used. In some cases, Bazett's formula over-corrects at high heart rates and under-corrects at low heart rates but is reasonably accurate for heart rates between 60 and 100 bpm. In some cases, the Friderecia's formula or the Framingham formula may be more useful at heart rate outside this range.

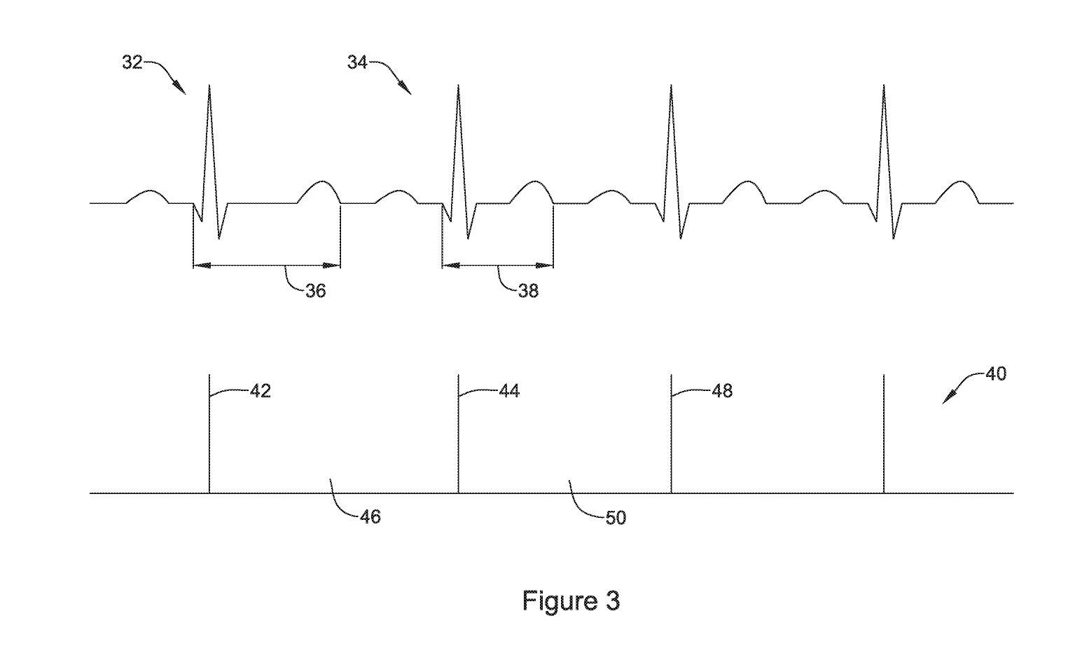

FIG. 3 shows an example ECG trace for several heartbeat, including a first QRS interval 32 and a second QRS interval 34. In this example, it should be recognized that the second QRS interval 34 may be immediately after the first QRS interval 32, as illustrated. In some cases, there may be one or more intervening QRS intervals (not shown). It is important to note that the first QRS interval 32 defines a QT interval 36 while the second QRS interval 34 defines a QT interval 38. As shown, the QT interval 38 is shorter in duration than the QT interval 36. In FIG. 3, a series 40 of pacing pulses are also shown. A pacing pulse 42 and a subsequent pacing pulse 44 are spaced apart by a first pacing interval 46. A subsequent pacing pulse 48 is spaced apart from the pacing pulse 44 by a second pacing interval 50. It can be seen that the second pacing interval 50 is shorter than the first pacing interval 46. In this example, this corresponds to the QT interval 38 being shorter than the QT interval 36. The difference between the QT interval 36 and the shorter QT interval 38 may represent an increase in cardiac load, thus necessitating an increase in the pacing rate as indicated by the difference between the second pacing interval 50 and the first pacing interval 46. In some cases, a single QT interval of shorter duration, such as the QT interval 38, may be used as an indication of increased cardiac load. In some cases, a single QT interval that has changed, or even several successive QT intervals, may be ignored as transient events. In some instances, a rolling average of QT intervals, for example, may be used as an indication of a changing cardiac load in order to identify a changing cardiac load while not immediately reacting to what may otherwise be a transient change or an aberrant signal.

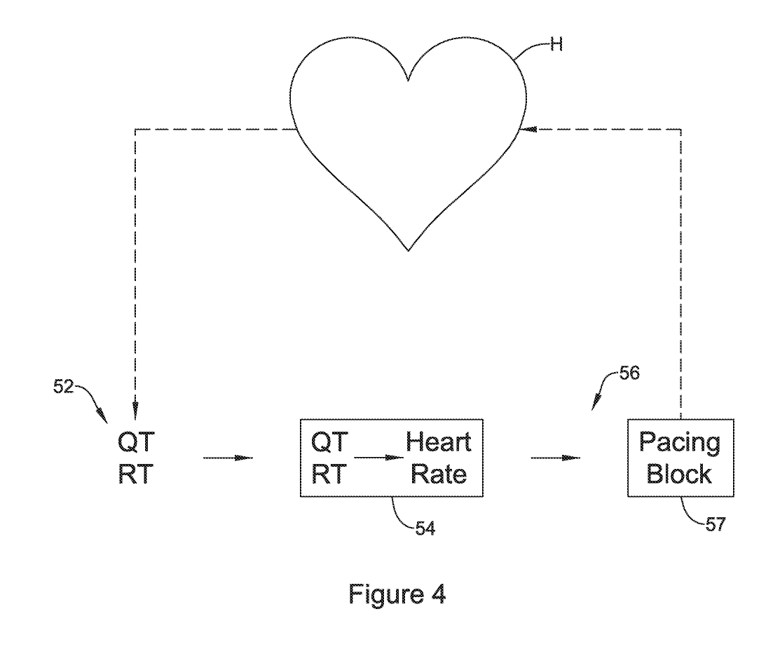

FIGS. 4 through 7 show illustrative but non-limiting examples of pacing control algorithms or systems in which a QT interval, or a related parameter such as the RT interval, is used to at least partially determine a cardiac pacing rate. In some cases, these pacing control algorithms or systems may be at least partially implemented in an implanted medical device such as an LCP or other pacemaker. In some instances, portions of these pacing control algorithms or systems may be implemented in an LCP while other portions of these pacing control algorithms or systems may be implemented in another implanted medical device such as a subcutaneous implantable cardioverter-defibrillator (SICD) that is in operative communication with an LCP.

In FIG. 4, a QT or RT parameter 52 is obtained from a heart H. For example, an implanted medical device may include several electrodes that may be utilized to sense a signal representing the QT or RT parameter 52. The QT or RT parameter 52 may be converted into a heart rate 56 by a conversion block 54 using any of a variety of different conversion mechanisms such as, but not limited to, those discussed above with respect to FIG. 2. A pacing block 57 may receive desired heart rate 56 from the conversion block 54 and may pace the heart H at the heart rate 56. While shown schematically, it will be appreciated that the conversion block 54 may be manifested in hardware and/or software and may be disposed within a single device or its functionality may, for example, be divided between two or more implanted or even external devices.

In some cases, using the QT (or RT) interval to determine a pacing heart rate may suffer from positive reinforcement or feedback. For example, and under some conditions, an increased pacing rate by the conversion block 54 may cause the QT interval to shorten, which then may cause the conversion block 54 to further increase the pacing rate. This can create a runaway situation. To help address such a runaway situation, it is contemplated that a governor may be applied to the pacing rate.

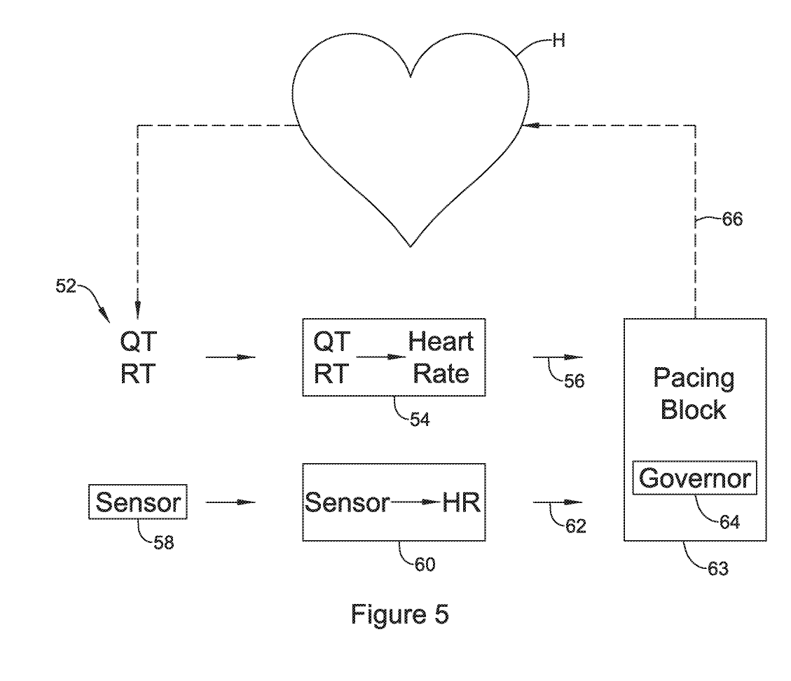

FIG. 5 illustrates a pacing control algorithm or system in which a QT or RT parameter 52 is obtained from a heart H and is converted into a heart rate 56 by conversion block 54. The heart rate 56 may be used to pace the heart H by a pacing block 63. In some cases, the QT or RT parameter 52 may be considered a first measure of cardiac load. A sensor 58 may also be configured to detect or otherwise determine a second parameter related to cardiac load of the heart H, which can then be used to determine, and sometimes limit, the pacing rate of the heart H. The sensor 58 may be any of a variety of different sensors, including but not limited to an accelerometer, a gyroscope, a respiration sensor, a heart sounds sensor and/or the like.

In the example shown in FIG. 5, the output from the sensor 58 passes to a conversion block 60 which provides an output 62. Output 62 may, for example, be a heart rate or other measure of cardiac load as determined by the conversion block 60. While shown schematically, it will be appreciated that the conversion block 60 may be manifested in hardware and/or software and may be disposed within a single device or its functionality may, for example, be divided between two or more implanted or even external devices. The heart rate 56 determined by conversion block 54 and the output 62 determined by conversion block 60 may be provided to a pacing block 63 with a governor 64. The governor 64, which may also be manifested in hardware and/or software, may perform a comparison of the inputs ix) 56 and 62. In some cases, the input 62 may be compared to the input 56 to determine if the input 56 is appropriate for pacing the heart H (e.g. the pacing rate determined from the QT or RT interval by conversion block 54 is appropriate for the activity level sensed by sensor 58). In some instances, the input 56 and the input 62 may be subjected to some mathematical operations by the governor 64 to provide a blended pacing rate that is used by the pacing block 63 to pace the heart H.

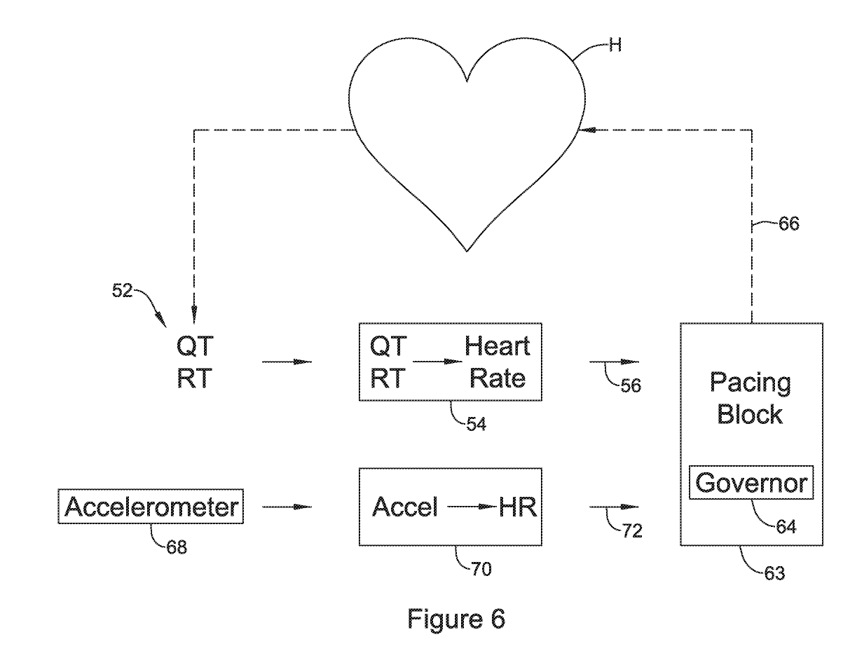

FIG. 6 illustrates another pacing control algorithm or system that includes a governor 64. A QT or RT parameter 52 is obtained from a heart H and is converted into a corresponding heart rate 56 by conversion block 54. The heart rate 56 may then be used to pace the heart H. An accelerometer 68 may be temporarily turned on in order to detect or otherwise determine a second parameter that may be used to help determine an appropriate pacing rate. In some cases, the QT or RT parameter 52 may be considered as being a first measure of cardiac load while an output from the accelerometer 68 may be considered as being a second measure of cardiac load.

In the example shown, the output from the accelerometer 68 passes to a conversion block 70 which provides an output 72. Output 72 may, for example, be a heart rate determined by the conversion block 70, or some other measure of cardiac load of the heart H. While shown schematically, it will be appreciated that the conversion block 70 may be manifested in hardware and/or software and may be disposed within a single device or its functionality may, for example, be divided between two or more implanted or even external devices. The heart rate 56 and the output 72 are shown provided to the governor 64 of pacing block 63. The governor 64, which may also be manifested in hardware and/or software, determines a pacing rate for the pacing block 63 to pace the heart H based at least in part on the inputs 56 and 72. In some cases, the governor 64 may compare the input 56 to the input 72 to determine if the input 56 is appropriate for pacing the heart H (e.g. the pacing rate determined from the QT or RT interval by conversion block 54 is appropriate for the activity level sensed by accelerometer 68). In some instances, the input 56 and the input 72 may be subjected to some mathematical operations by the governor 64 to provide a blended pacing rate that is used by the pacing block 63 to pace the heart H.

When the accelerometer 68 is positioned in an LCP that is attached to a wall (e.g. ventricle wall) of the heart H, the accelerometer 68 may be activated or sampled during quiet period between active heart beats. This may help reduce the contribution of heart contractions to the sensed activity level of the patient. This may be useful when it is desirable to sense a measure of gross movement of the patient (e.g. posture changes, walking, running, going up stairs, etc.). Alternatively, or in addition, the accelerometer 68 may be activated or sampled during the heart contractions. This may be useful to determined, for example, a time derivative of the left ventricular pressure (DP/dt), which can be an indication of cardiac load and thus patient activity. These are just some examples.

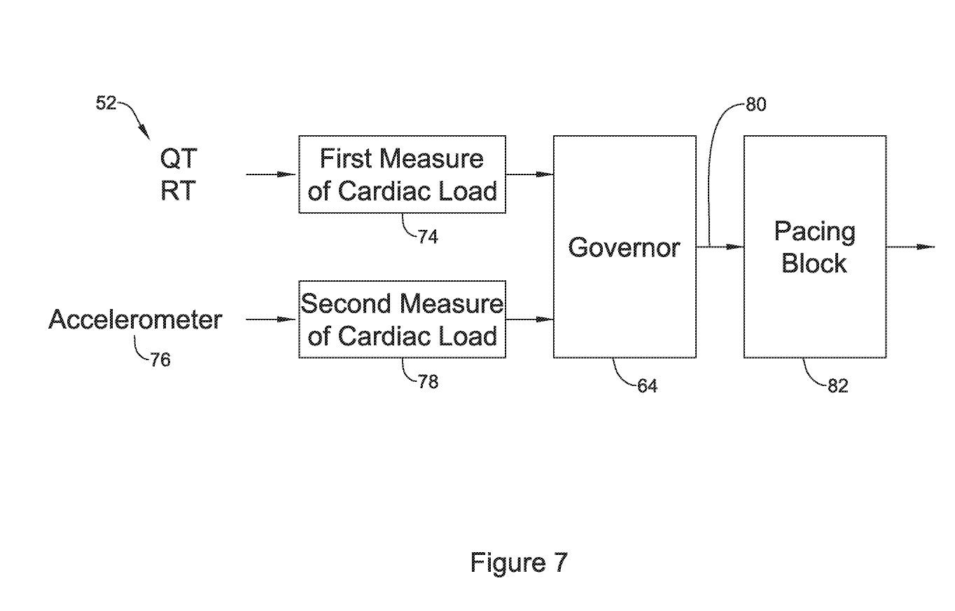

FIG. 7 schematically illustrates a pacing control algorithm or system in which a QT or RT parameter 52 is used to determine a first measure of cardiac load, as indicated at block 74, and an accelerometer parameter 76 is used to determine a second measure of cardiac load, as indicated at block 78. The first measure of cardiac load and the second measure of cardiac load are provided to a governor 64. The governor 64, which may be manifested in hardware and/or software, may perform a comparison of some sort on the first measure of cardiac load and the second measure of cardiac load. In some cases, the second measure of cardiac load may be compared to the first measure of cardiac load to determine if the first measure of cardiac load is appropriate for pacing the heart H. In some instances, the first measure of cardiac load and the second measure of cardiac load may be subjected to some mathematical operations that provide an output 80 that subsequently is fed into a pacing block 82, which produces a pacing rate for pacing the heart H. In some cases, the pacing block 82 may provide a further check on the output of the governor 64. For example, the pacing block 82 may keep track of the pacing rate over time, and may make adjustments if appropriate. In some cases, the pacing block 82 may provide upper and lower cutoff limits to the pacing rate, and will keep the pacing rate between the upper and lower pacing rate limits.

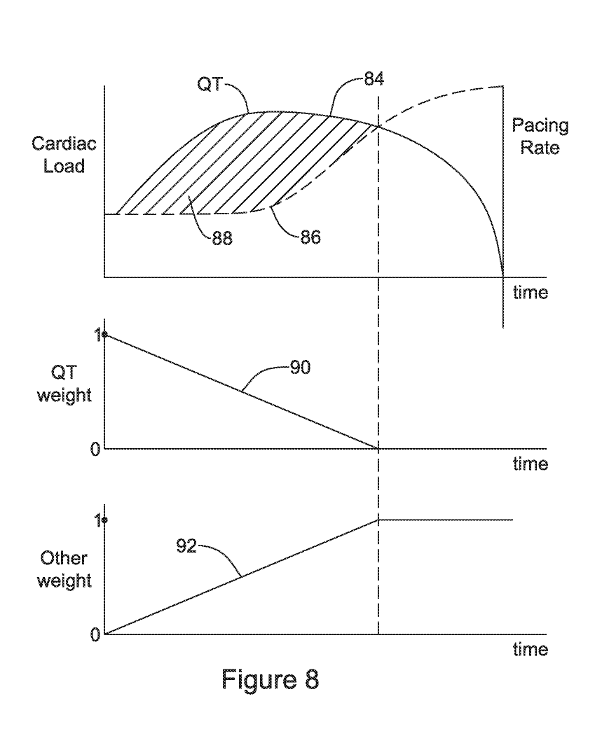

It will be appreciated that some measures of cardiac load may provide an instantaneous or nearly instantaneous indication that cardiac load has change, either in a positive direction or in a negative direction. Some measures of cardiac load may provide a delayed indication. For example, the QT interval (or RT interval) may respond almost immediately to a change in cardiac load, while other measures, such as impedance, respiration rate, blood temperature and the like may be delayed. In some embodiments, the first measure of cardiac load may be considered as being a rapidly responding measure of cardiac load and the second measure of cardiac load may be considered as being a more slowly responding measure of cardiac load. In some cases, the first measure of cardiac load may be thought of as being useful in determining an appropriate pacing rate in the very first seconds after an increase in cardiac load, before the second measure of cardiac load has caught up to the increased cardiac load. This is illustrated in FIG. 8.

FIG. 8 provides several graphs, each with time along the x-axis. The first graph shows cardiac load versus time. Ideally, the pacing rate should largely track the cardiac load. A line 84 shows how a first measure of cardiac load, such as the QT (or RT) interval, responds to a change in cardiac load over time. It can be seen that the first measure of cardiac load responds quickly. A line 86 shows how a second measure of cardiac load, such as impedance (respiration), blood temperature or the like, responds to the same change in cardiac load over time. It can be seen that the second measure of cardiac load responds more slowly. An area 88, defined between the line 84 and the line 86, may be considered as representing a potential improvement in determining an appropriate pacing rate to match the pacing rate to the actual cardiac load of the heart H, particularly in response to sudden changes in cardiac load.

In some cases, the relative importance of the first measure of cardiac load and the second measure of cardiac load may vary over time. For example, and as shown in the second graph, the first measure of cardiac load, such as the QT or RT interval, may initially have a weight of about 1 (on a scale of 0 to 1) and may decrease over time until such time as the second measure of cardiac load has caught up. This can be seen in the line 90 in the second graph. Corresponding, as shown in the third graph and line 92, the second measure of cardiac load, such as impedance, blood temperature, or the like, may initially have a weight of about 0, and may increase over time to about 1. While the line 90 and the line 92 are illustrated as being linear, it will be appreciated that in some cases one or both of the lines 90 and 92 may instead be represented by non-linear equations, for example.

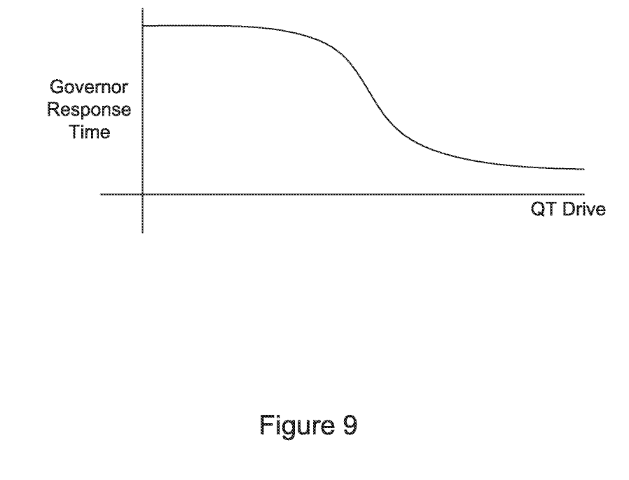

It will be appreciated that FIG. 8 provides an example of how the governor 64 may determine a desired pacing rate. In some cases, and as shown for example in FIG. 9, the speed at which the governor 64 responds to a change in the QT (or RT) interval may depend at least in part upon the magnitude of the QT (or RT) value. FIG. 9 plots governor response time versus QT drive. QT drive will be appreciated to be related to the inverse of the QT interval. A smaller QT interval represents an increased QT drive. In some cases, and with reference to FIGS. 4-6, the QT drive may correspond to the output 56 of conversion block 54. The governor 64 may simply use the QT drive to determine a desired pacing rate until the QT drive signal becomes elevated. In some cases, the governor 64 may be configured to sample the second measure of cardiac load to check the QT drive more often as the QT drive increases, as shown in FIG. 9. This may help save battery energy by not requiring the sensor 58 (e.g. accelerometer) to be activated and sampled as often when the QT drive is low (e.g. patient is sleeping or sitting).

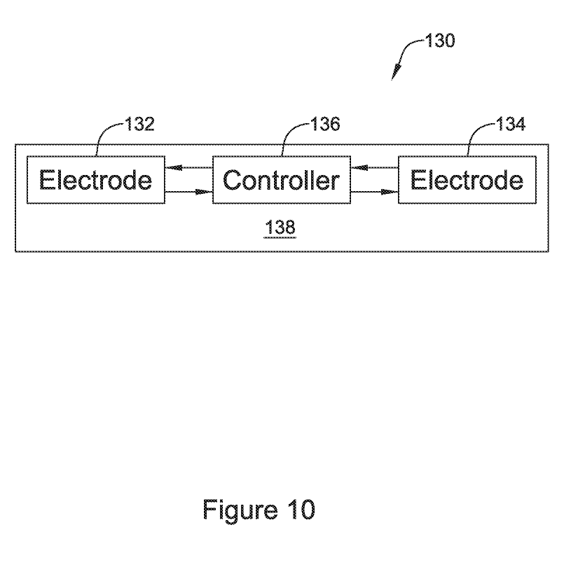

FIGS. 10-12 are schematic illustrations of LCPs that may be configured to sense and pace a patient's heart. In FIG. 10, an LCP 130 includes an electrode 132 and an electrode 134. In some cases, the LCP 130 may include additional electrodes (not illustrated). The electrodes 132 and 134 may be configured to receive electrical signals emanating from the patient's heart. A controller 136 may be disposed within a housing 138. The controller 136 may be coupled to the electrodes 132, 134 and may be configured to ascertain a first measure of cardiac load based at least in part upon electrical signals received by the electrodes 132 and 134. In some cases, the controller 136 may be configured to receive a second measure of cardiac load. The second measure of cardiac load may be measured or otherwise detected by the LCP 130, or may be measured or detected remotely and transmitted to the LCP 130. The controller 136 may be configured to determine a pacing rate based at least in part upon the first measure of cardiac load and the second measure of cardiac load and to pace the patient's heart in accordance with the determined pacing rate. In some cases, the first measure of cardiac load includes a measure that responds more quickly to changes in cardiac activity than the second measure of cardiac load.

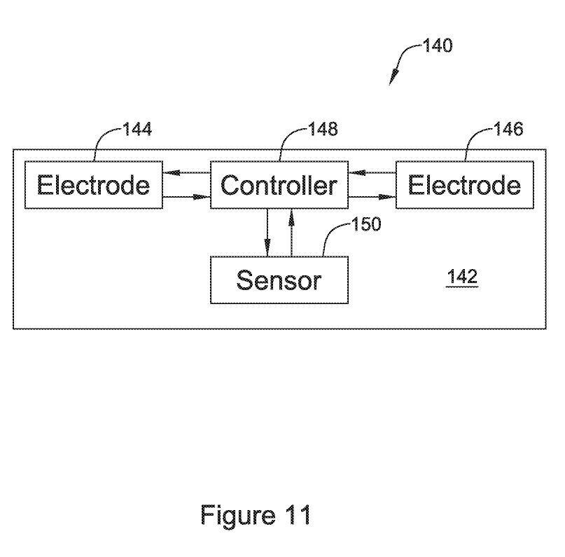

FIG. 11 illustrates an LCP 140 that includes a housing 142. A first electrode 144 and a second electrode 146 are secured relative to the housing 142 and are configured to receive electrical signals emanating from the patient's heart. While two electrodes 144, 146 are illustrated, it will be appreciated that the LCP 140 may include additional electrodes. A controller 148 may be coupled to the electrodes 144, 146 such that the controller may receive electrical signals from the electrodes 144, 146. The controller 148 may determine a first measure of cardiac load based on the received electrical signals. In some instances, the first measure of cardiac load is representative of how quickly the patient's heart prepares for a subsequent heartbeat. For example, in some cases, the first measure of cardiac load is representative of a time required for ventricle depolarization and repolarization. In some cases, the first measure of cardiac load includes an RT interval. In some instances, the controller 148 is configured to temporarily ignore any changes in the first measure of cardiac load, for at least two heartbeats, in determining the pacing rate. In some cases, the controller 148 may be configured to determine a plurality of first measures of cardiac load over time based on the received electrical signals, and to determine the pacing rate based on a moving average of two or more of the plurality of first measures of cardiac load.

A sensor 150 is configured to provide a second measure of cardiac load and is operatively coupled to the controller 148. In some cases, the second measure of cardiac load is representative of gross movement of the patient, although this is not required. In some cases, the sensor 150 is an accelerometer, and the second measure of cardiac load is derived at least in part from a signal outputted from the accelerometer. In some instances, the controller 148 determines a pacing rate based at least in part upon the first measure of cardiac load and paces the patient's heart in accordance with the determined pacing rate. The controller 148 may use the second measure of cardiac load to determine a pacing rate limit, and to limit the determined pacing rate based on the pacing rate limit.

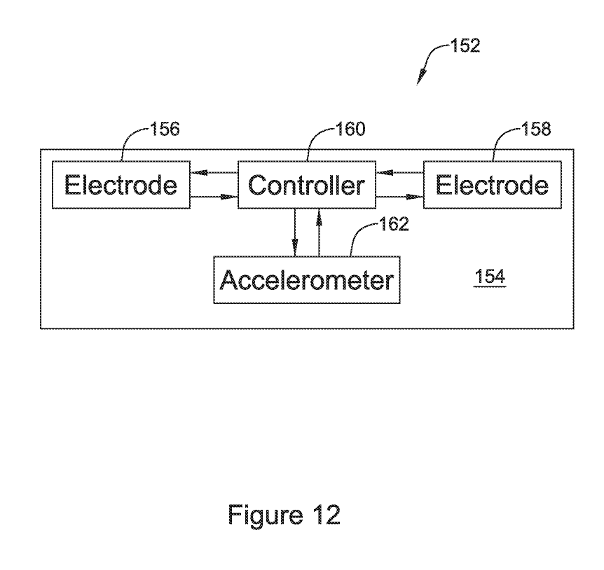

FIG. 12 illustrates an LCP 152 that includes a housing 154. A first electrode 156 and a second electrode 158 are secured relative to the housing 154 and are configured to receive electrical signals emanating from the patient's heart. While two electrodes 156, 158 are illustrated, it will be appreciated that the LCP 152 may include additional electrodes. A controller 160 may be coupled to the electrodes 156, 158 such that the controller 160 may receive electrical signals from the electrodes 156, 158. The controller 160 may be configured to receive electrical signals from the electrodes 156, 158 indicative of a heartbeat of the patient's heart, the received electrical signals including one or more of a Q, an R, and an S feature, as well as a T feature of a QRST complex.

The controller 160 may be configured to determine a first measure of cardiac load based at least in part on a delay between the Q or R feature and the T feature for the heartbeat. In some instances, the controller 160 may be configured to determine the first measure of cardiac load based at least in part on a delay between the R feature and the T feature for the heartbeat. In some cases, the controller 160 may be configured to receive electrical signals from the electrodes 156, 158 that are indicative of a plurality of heartbeats, and may be configured to determine the first measure of cardiac load based at least in part on a delay between the Q or R feature and the T feature of each of two or more heartbeats.

The LCP 152 includes an accelerometer 162 that is configured to sense an activity level of the patient. The controller 160 may be configured to determine a second measure of cardiac load based at least in part on the activity level of the patient as indicated by the accelerometer 162. In some cases, the controller 160 is configured to repeatedly activate and deactivate the accelerometer to intermittently sense the activity level of the patient and to determine the second measure of cardiac load based at least in part on two or more intermittently sensed activity levels of the patient. In some cases, the accelerometer 162 may be configured to sense non-cardiac movement of the patient, and the activity level of the patient is based on the sensed non-cardiac movement of the patient. In some instances, the accelerometer 162 may be configured to sense cardiac movement, and the activity level of the patient is based on the sensed cardiac movement. The accelerometer 162 may, in some cases, be configured to sense non-cardiac movement of the patient and cardiac movement, and the activity level of the patient is based at least in part on the sensed non-cardiac movement of the patient and the sensed cardiac movement.

In some cases, the controller 160 may be configured to determine a pacing rate based at least in part upon the first measure of cardiac load and to pace the patient's heart in accordance with the determined pacing rate. The controller 160 may be configured to utilize the second measure of cardiac load to act as a governor to the determined pacing rate. In some instances, the controller 160 may be configured to determine the pacing rate based at least in part upon the first measure of cardiac load using a linear function having a non-zero slope. In some cases, the controller 160 may be configured to act as a governor by determining a pacing rate limit based at least in part on the second measure of cardiac load, and to limit the determined pacing rate based on the pacing rate limit. In some instances, the pacing rate limit is a function of the second measure of cardiac load, and the function is not a constant function.

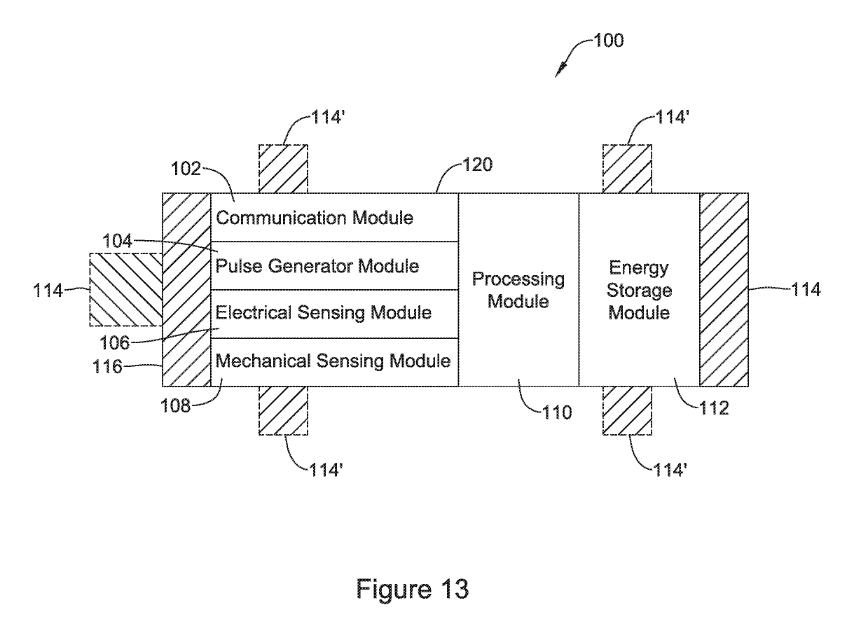

FIGS. 13-16 shown some illustrative systems and devices that are suitable for carrying out the present disclosure. FIG. 13 is a conceptual schematic block diagram of an exemplary leadless cardiac pacemaker (LCP) that may be implanted on the heart or within a chamber of the heart and may operate to sense physiological signals and parameters and deliver one or more types of electrical stimulation therapy to the heart of the patient, including using a first measure of cardiac load and/or a second measure of cardiac load to determine a pacing rate. Example electrical stimulation therapy may include bradycardia pacing, rate responsive pacing therapy, cardiac resynchronization therapy (CRT), anti-tachycardia pacing (ATP) therapy and/or the like. As can be seen in FIG. 13, LCP 100 may be a compact device with all components housed within LCP 100 or directly on housing 120. In some instances, LCP 100 may include communication module 102, pulse generator module 104, electrical sensing module 106, mechanical sensing module 108, processing module 110, energy storage module 112, and electrodes 114.

As depicted in FIG. 13, LCP 100 may include electrodes 114, which can be secured relative to housing 120 and electrically exposed to tissue and/or blood surrounding LCP 100. Electrodes 114 may generally conduct electrical signals to and from LCP 100 and the surrounding tissue and/or blood. Such electrical signals can include communication signals, electrical stimulation pulses, and intrinsic cardiac electrical signals, to name a few. Intrinsic cardiac electrical signals may include electrical signals generated by the heart and may be represented by an electrocardiogram (ECG).

Electrodes 114 may include one or more biocompatible conductive materials such as various metals or alloys that are known to be safe for implantation within a human body. In some instances, electrodes 114 may be generally disposed on either end of LCP 100 and may be in electrical communication with one or more of modules 102, 104, 106, 108, and 110. In embodiments where electrodes 114 are secured directly to housing 120, an insulative material may electrically isolate the electrodes 114 from adjacent electrodes, housing 120, and/or other parts of LCP 100. In some instances, some or all of electrodes 114 may be spaced from housing 120 and connected to housing 120 and/or other components of LCP 100 through connecting wires. In such instances, the electrodes 114 may be placed on a tail (not shown) that extends out away from the housing 120. As shown in FIG. 1, in some embodiments, LCP 100 may include electrodes 114′. Electrodes 114′ may be in addition to electrodes 114, or may replace one or more of electrodes 114. Electrodes 114′ may be similar to electrodes 114 except that electrodes 114′ are disposed on the sides of LCP 100. In some cases, electrodes 114′ may increase the number of electrodes by which LCP 100 may deliver communication signals and/or electrical stimulation pulses, and/or may sense intrinsic cardiac electrical signals, communication signals, and/or electrical stimulation pulses.

Electrodes 114 and/or 114′ may assume any of a variety of sizes and/or shapes, and may be spaced at any of a variety of spacings. For example, electrodes 114 may have an outer diameter of two to twenty millimeters (mm). In other embodiments, electrodes 114 and/or 114′ may have a diameter of two, three, five, seven millimeters (mm), or any other suitable diameter, dimension and/or shape. Example lengths for electrodes 114 and/or 114′ may include, for example, one, three, five, ten millimeters (mm), or any other suitable length. As used herein, the length is a dimension of electrodes 114 and/or 114′ that extends away from the outer surface of the housing 120. In some instances, at least some of electrodes 114 and/or 114′ may be spaced from one another by a distance of twenty, thirty, forty, fifty millimeters (mm), or any other suitable spacing. The electrodes 114 and/or 114′ of a single device may have different sizes with respect to each other, and the spacing and/or lengths of the electrodes on the device may or may not be uniform.

In the embodiment shown, communication module 102 may be electrically coupled to electrodes 114 and/or 114′ and may be configured to deliver communication pulses to tissues of the patient for communicating with other devices such as sensors, programmers, other medical devices, and/or the like. Communication signals, as used herein, may be any modulated signal that conveys information to another device, either by itself or in conjunction with one or more other modulated signals. In some embodiments, communication signals may be limited to sub-threshold signals that do not result in capture of the heart yet still convey information. The communication signals may be delivered to another device that is located either external or internal to the patient's body. In some instances, the communication may take the form of distinct communication pulses separated by various amounts of time. In some of these cases, the timing between successive pulses may convey information. Communication module 102 may additionally be configured to sense for communication signals delivered by other devices, which may be located external or internal to the patient's body.

Communication module 102 may communicate to help accomplish one or more desired functions. Some example functions include delivering sensed data, using communicated data for determining occurrences of events such as arrhythmias, coordinating delivery of electrical stimulation therapy, and/or other functions. In some cases, LCP 100 may use communication signals to communicate raw information, processed information, messages and/or commands, and/or other data. Raw information may include information such as sensed electrical signals (e.g. a sensed ECG), signals gathered from coupled sensors, and the like. In some embodiments, the processed information may include signals that have been filtered using one or more signal processing techniques. Processed information may also include parameters and/or events that are determined by the LCP 100 and/or another device, such as a determined heart rate, timing of determined heartbeats, timing of other determined events, determinations of threshold crossings, expirations of monitored time periods, accelerometer signals, activity level parameters, blood-oxygen parameters, blood pressure parameters, heart sound parameters, and the like. Messages and/or commands may include instructions or the like directing another device to take action, notifications of imminent actions of the sending device, requests for reading from the receiving device, requests for writing data to the receiving device, information messages, and/or other messages commands.

In at least some embodiments, communication module 102 (or LCP 100) may further include switching circuitry to selectively connect one or more of electrodes 114 and/or 114′ to communication module 102 in order to select which electrodes 114 and/or 114′ that communication module 102 delivers communication pulses. It is contemplated that communication module 102 may be communicating with other devices via conducted signals, radio frequency (RF) signals, optical signals, acoustic signals, inductive coupling, and/or any other suitable communication methodology. Where communication module 102 generates electrical communication signals, communication module 102 may include one or more capacitor elements and/or other charge storage devices to aid in generating and delivering communication signals. In the embodiment shown, communication module 102 may use energy stored in energy storage module 112 to generate the communication signals. In at least some examples, communication module 102 may include a switching circuit that is connected to energy storage module 112 and, with the switching circuitry, may connect energy storage module 112 to one or more of electrodes 114/114′ to generate the communication signals.

As shown in FIG. 13, a pulse generator module 104 may be electrically connected to one or more of electrodes 114 and/or 114′. Pulse generator module 104 may be configured to generate electrical stimulation pulses and deliver the electrical stimulation pulses to tissues of a patient via one or more of the electrodes 114 and/or 114′ in order to effectuate one or more electrical stimulation therapies. Electrical stimulation pulses as used herein are meant to encompass any electrical signals that may be delivered to tissue of a patient for purposes of treatment of any type of disease or abnormality. For example, when used to treat heart disease, the pulse generator module 104 may generate electrical stimulation pacing pulses for capturing the heart of the patient, i.e. causing the heart to contract in response to the delivered electrical stimulation pulse. In some of these cases, LCP 100 may vary the rate at which pulse generator module 104 generates the electrical stimulation pulses, for example in rate adaptive pacing. In other embodiments, the electrical stimulation pulses may include defibrillation/cardioversion pulses for shocking the heart out of fibrillation or into a normal heart rhythm. In yet other embodiments, the electrical stimulation pulses may include anti-tachycardia pacing (ATP) pulses. It should be understood that these are just some examples. When used to treat other ailments, the pulse generator module 104 may generate electrical stimulation pulses suitable for neurostimulation therapy or the like. Pulse generator module 104 may include one or more capacitor elements and/or other charge storage devices to aid in generating and delivering appropriate electrical stimulation pulses. In at least some embodiments, pulse generator module 104 may use energy stored in energy storage module 112 to generate the electrical stimulation pulses. In some particular embodiments, pulse generator module 104 may include a switching circuit that is connected to energy storage module 112 and may connect energy storage module 112 to one or more of electrodes 114/114′ to generate electrical stimulation pulses.

LCP 100 may further include an electrical sensing module 106 and mechanical sensing module 108. Electrical sensing module 106 may be configured to sense intrinsic cardiac electrical signals conducted from electrodes 114 and/or 114′ to electrical sensing module 106. For example, electrical sensing module 106 may be electrically connected to one or more electrodes 114 and/or 114′ and electrical sensing module 106 may be configured to receive cardiac electrical signals conducted through electrodes 114 and/or 114′ via a sensor amplifier or the like. In some embodiments, the cardiac electrical signals may represent local information from the chamber in which LCP 100 is implanted. For instance, if LCP 100 is implanted within a ventricle of the heart, cardiac electrical signals sensed by LCP 100 through electrodes 114 and/or 114′ may represent ventricular cardiac electrical signals. Mechanical sensing module 108 may include, or be electrically connected to, various sensors, such as accelerometers, including multi-axis accelerometers such as two- or three-axis accelerometers, gyroscopes, including multi-axis gyroscopes such as two- or three-axis gyroscopes, blood pressure sensors, heart sound sensors, piezoelectric sensors, blood-oxygen sensors, and/or other sensors which measure one or more physiological parameters of the heart and/or patient. Mechanical sensing module 108, when present, may gather signals from the sensors indicative of the various physiological parameters. Both electrical sensing module 106 and mechanical sensing module 108 may be connected to processing module 110 and may provide signals representative of the sensed cardiac electrical signals and/or physiological signals to processing module 110. Although described with respect to FIG. 13 as separate sensing modules, in some embodiments, electrical sensing module 106 and mechanical sensing module 108 may be combined into a single module. In at least some examples, LCP 100 may only include one of electrical sensing module 106 and mechanical sensing module 108. In some cases, any combination of the processing module 110, electrical sensing module 106, mechanical sensing module 108, communication module 102, pulse generator module 104 and/or energy storage module may be considered a controller of the LCP 100.

Processing module 110 may be configured to direct the operation of LCP 100 and may, in some embodiments, be termed a controller. For example, processing module 110 may be configured to receive cardiac electrical signals from electrical sensing module 106 and/or physiological signals from mechanical sensing module 108. Based on the received signals, processing module 110 may determine, for example, occurrences and types of arrhythmias and other determinations such as whether LCP 100 has become dislodged. Processing module 110 may further receive information from communication module 102. In some embodiments, processing module 110 may additionally use such received information to determine occurrences and types of arrhythmias and/or and other determinations such as whether LCP 100 has become dislodged. In still some additional embodiments, LCP 100 may use the received information instead of the signals received from electrical sensing module 106 and/or mechanical sensing module 108—for instance if the received information is deemed to be more accurate than the signals received from electrical sensing module 106 and/or mechanical sensing module 108 or if electrical sensing module 106 and/or mechanical sensing module 108 have been disabled or omitted from LCP 100.

After determining an occurrence of an arrhythmia, processing module 110 may control pulse generator module 104 to generate electrical stimulation pulses in accordance with one or more electrical stimulation therapies to treat the determined arrhythmia. For example, processing module 110 may control pulse generator module 104 to generate pacing pulses with varying parameters and in different sequences to effectuate one or more electrical stimulation therapies. As one example, in controlling pulse generator module 104 to deliver bradycardia pacing therapy, processing module 110 may control pulse generator module 104 to deliver pacing pulses designed to capture the heart of the patient at a regular interval to help prevent the heart of a patient from falling below a predetermined threshold. In some cases, the rate of pacing may be increased with an increased activity level of the patient (e.g. rate adaptive pacing). For instance, processing module 110 may monitor one or more physiological parameters of the patient which may indicate a need for an increased heart rate (e.g. due to increased metabolic demand). Processing module 110 may then increase the rate at which pulse generator module 104 generates electrical stimulation pulses. Adjusting the rate of delivery of the electrical stimulation pulses based on the one or more physiological parameters may extend the battery life of LCP 100 by only requiring higher rates of delivery of electrical stimulation pulses when the physiological parameters indicate there is a need for increased cardiac output. Additionally, adjusting the rate of delivery of the electrical stimulation pulses may increase a comfort level of the patient by more closely matching the rate of delivery of electrical stimulation pulses with the cardiac output need of the patient.

For ATP therapy, processing module 110 may control pulse generator module 104 to deliver pacing pulses at a rate faster than an intrinsic heart rate of a patient in attempt to force the heart to beat in response to the delivered pacing pulses rather than in response to intrinsic cardiac electrical signals. Once the heart is following the pacing pulses, processing module 110 may control pulse generator module 104 to reduce the rate of delivered pacing pulses down to a safer level. In CRT, processing module 110 may control pulse generator module 104 to deliver pacing pulses in coordination with another device to cause the heart to contract more efficiently. In cases where pulse generator module 104 is capable of generating defibrillation and/or cardioversion pulses for defibrillation/cardioversion therapy, processing module 110 may control pulse generator module 104 to generate such defibrillation and/or cardioversion pulses. In some cases, processing module 110 may control pulse generator module 104 to generate electrical stimulation pulses to provide electrical stimulation therapies different than those examples described above.

Aside from controlling pulse generator module 104 to generate different types of electrical stimulation pulses and in different sequences, in some embodiments, processing module 110 may also control pulse generator module 104 to generate the various electrical stimulation pulses with varying pulse parameters. For example, each electrical stimulation pulse may have a pulse width and a pulse amplitude. Processing module 110 may control pulse generator module 104 to generate the various electrical stimulation pulses with specific pulse widths and pulse amplitudes. For example, processing module 110 may cause pulse generator module 104 to adjust the pulse width and/or the pulse amplitude of electrical stimulation pulses if the electrical stimulation pulses are not effectively capturing the heart. Such control of the specific parameters of the various electrical stimulation pulses may help LCP 100 provide more effective delivery of electrical stimulation therapy.

In some embodiments, processing module 110 may further control communication module 102 to send information to other devices. For example, processing module 110 may control communication module 102 to generate one or more communication signals for communicating with other devices of a system of devices. For instance, processing module 110 may control communication module 102 to generate communication signals in particular pulse sequences, where the specific sequences convey different information. Communication module 102 may also receive communication signals for potential action by processing module 110.

In further embodiments, processing module 110 may control switching circuitry by which communication module 102 and pulse generator module 104 deliver communication signals and/or electrical stimulation pulses to tissue of the patient. As described above, both communication module 102 and pulse generator module 104 may include circuitry for connecting one or more electrodes 114 and/114′ to communication module 102 and/or pulse generator module 104 so those modules may deliver the communication signals and electrical stimulation pulses to tissue of the patient. The specific combination of one or more electrodes by which communication module 102 and/or pulse generator module 104 deliver communication signals and electrical stimulation pulses may influence the reception of communication signals and/or the effectiveness of electrical stimulation pulses. Although it was described that each of communication module 102 and pulse generator module 104 may include switching circuitry, in some embodiments, LCP 100 may have a single switching module connected to the communication module 102, the pulse generator module 104, and electrodes 114 and/or 114′. In such embodiments, processing module 110 may control the switching module to connect modules 102/104 and electrodes 114/114′ as appropriate.

In some embodiments, processing module 110 may include a pre-programmed chip, such as a very-large-scale integration (VLSI) chip or an application specific integrated circuit (ASIC). In such embodiments, the chip may be pre-programmed with control logic in order to control the operation of LCP 100. By using a pre-programmed chip, processing module 110 may use less power than other programmable circuits while able to maintain basic functionality, thereby potentially increasing the battery life of LCP 100. In other instances, processing module 110 may include a programmable microprocessor or the like. Such a programmable microprocessor may allow a user to adjust the control logic of LCP 100 after manufacture, thereby allowing for greater flexibility of LCP 100 than when using a pre-programmed chip. In still other embodiments, processing module 110 may not be a single component. For example, processing module 110 may include multiple components positioned at disparate locations within LCP 100 in order to perform the various described functions. For example, certain functions may be performed in one component of processing module 110, while other functions are performed in a separate component of processing module 110.

Processing module 110, in additional embodiments, may include a memory circuit and processing module 110 may store information on and read information from the memory circuit. In other embodiments, LCP 100 may include a separate memory circuit (not shown) that is in communication with processing module 110, such that processing module 110 may read and write information to and from the separate memory circuit. The memory circuit, whether part of processing module 110 or separate from processing module 110, may be volatile memory, non-volatile memory, or a combination of volatile memory and non-volatile memory.

It is contemplated that the processing module 110 may, in some cases, implement in whole or in part, conversion blocks 54 and 60, pacing blocks 57, 63, 82, governor 64, and/or controller 136, 148, and 160 described above with respect to FIGS. 4-7 and 10-12.

Energy storage module 112 may provide a power source to LCP 100 for its operations. In some embodiments, energy storage module 112 may be a non-rechargeable lithium-based battery. In other embodiments, the non-rechargeable battery may be made from other suitable materials. In some embodiments, energy storage module 112 may include a rechargeable battery. In still other embodiments, energy storage module 112 may include other types of energy storage devices such as capacitors or super capacitors.

To implant LCP 100 inside a patient's body, an operator (e.g., a physician, clinician, etc.), may fix LCP 100 to the cardiac tissue of the patient's heart. To facilitate fixation, LCP 100 may include one or more anchors 116. The one or more anchors 116 are shown schematically in FIG. 1. The one or more anchors 116 may include any number of fixation or anchoring mechanisms. For example, one or more anchors 116 may include one or more pins, staples, threads, screws, helix, tines, and/or the like. In some embodiments, although not shown, one or more anchors 116 may include threads on its external surface that may run along at least a partial length of an anchor member. The threads may provide friction between the cardiac tissue and the anchor to help fix the anchor member within the cardiac tissue. In some cases, the one or more anchors 116 may include an anchor member that has a cork-screw shape that can be screwed into the cardiac tissue. In other embodiments, anchor 116 may include other structures such as barbs, spikes, or the like to facilitate engagement with the surrounding cardiac tissue.

In some examples, LCP 100 may be configured to be implanted on a patient's heart or within a chamber of the patient's heart. For instance, LCP 100 may be implanted within any of a left atrium, right atrium, left ventricle, or right ventricle of a patient's heart. By being implanted within a specific chamber, LCP 100 may be able to sense cardiac electrical signals originating or emanating from the specific chamber that other devices may not be able to sense with such resolution. Where LCP 100 is configured to be implanted on a patient's heart, LCP 100 may be configured to be implanted on or adjacent to one of the chambers of the heart, or on or adjacent to a path along which intrinsically generated cardiac electrical signals generally follow. In these examples, LCP 100 may also have an enhanced ability to sense localized intrinsic cardiac electrical signals and deliver localized electrical stimulation therapy. In embodiments where LCP 100 includes an accelerometer, LCP 100 may additionally be able to sense the motion of the cardiac wall to which LCP 100 is attached.

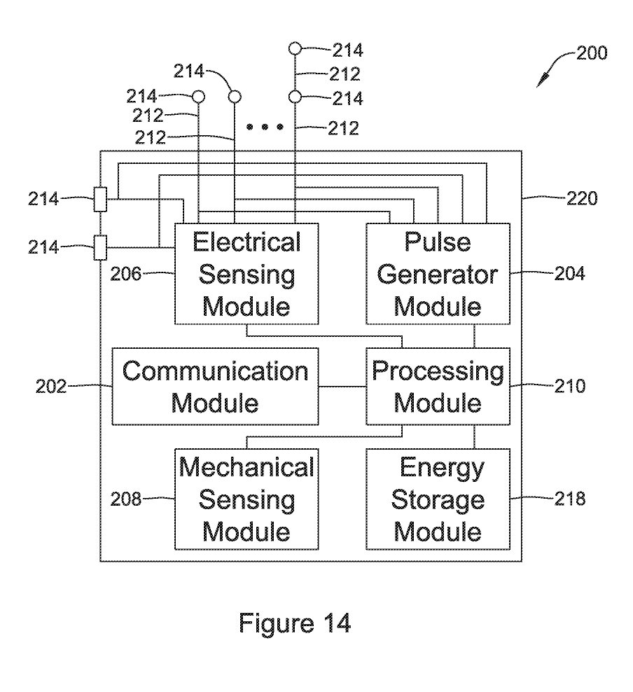

FIG. 14 depicts an embodiment of another device, medical device (MD) 200, which may operate to sense physiological signals and parameters and deliver one or more types of electrical stimulation therapy to tissues of the patient. In the embodiment shown, MD 200 may include a communication module 202, a pulse generator module 204, an electrical sensing module 206, a mechanical sensing module 208, a processing module 210, and an energy storage module 218. Each of modules 202, 204, 206, 208, and 210 may be similar to modules 102, 104, 106, 108, and 110 of LCP 100. Additionally, energy storage module 218 may be similar to energy storage module 112 of LCP 100. However, in some embodiments, MD 200 may have a larger volume within housing 220. In such embodiments, MD 200 may include a larger energy storage module 218 and/or a larger processing module 210 capable of handling more complex operations than processing module 110 of LCP 100.

While MD 200 may be another leadless device such as shown in FIG. 13, in some instances MD 200 may include leads, such as leads 212. Leads 212 may include electrical wires that conduct electrical signals between electrodes 214 and one or more modules located within housing 220. In some cases, leads 212 may be connected to and extend away from housing 220 of MD 200. In some embodiments, leads 212 are implanted on, within, or adjacent to a heart of a patient. Leads 212 may contain one or more electrodes 214 positioned at various locations on leads 212 and various distances from housing 220. Some leads 212 may only include a single electrode 214, while other leads 212 may include multiple electrodes 214. Generally, electrodes 214 are positioned on leads 212 such that when leads 212 are implanted within the patient, one or more of the electrodes 214 are positioned to perform a desired function. In some cases, the one or more of the electrodes 214 may be in contact with the patient's cardiac tissue. In other cases, the one or more of the electrodes 214 may be positioned subcutaneously but adjacent the patient's heart. The electrodes 214 may conduct intrinsically generated electrical cardiac signals to leads 212. Leads 212 may, in turn, conduct the received electrical cardiac signals to one or more of the modules 202, 204, 206, and 208 of MD 200. In some cases, MD 200 may generate electrical stimulation signals, and leads 212 may conduct the generated electrical stimulation signals to electrodes 214. Electrodes 214 may then conduct the electrical stimulation signals to the cardiac tissue of the patient (either directly or indirectly). MD 200 may also include one or more electrodes 214 not disposed on a lead 212. For example, one or more electrodes 214 may be connected directly to housing 220.

Leads 212, in some embodiments, may additionally contain one or more sensors, such as accelerometers, blood pressure sensors, heart sound sensors, blood-oxygen sensors, and/or other sensors which are configured to measure one or more physiological parameters of the heart and/or patient. In such embodiments, mechanical sensing module 208 may be in electrical communication with leads 212 and may receive signals generated from such sensors. While not required, in some embodiments MD 200 may be an implantable medical device. In such embodiments, housing 220 of MD 200 may be implanted in, for example, a transthoracic region of the patient. Housing 220 may generally include any of a number of known materials that are safe for implantation in a human body and may, when implanted, hermetically seal the various components of MD 200 from fluids and tissues of the patient's body. In such embodiments, leads 212 may be implanted at one or more various locations within the patient, such as within the heart of the patient, adjacent to the heart of the patient, adjacent to the spine of the patient, or any other desired location.

In some embodiments, MD 200 may be an implantable cardiac pacemaker (ICP). In these embodiments, MD 200 may have one or more leads, for example leads 212, which are implanted on or within the patient's heart. The one or more leads 212 may include one or more electrodes 214 that are in contact with cardiac tissue and/or blood of the patient's heart. MD 200 may be configured to sense intrinsically generated cardiac electrical signals and determine, for example, one or more cardiac arrhythmias based on analysis of the sensed signals. MD 200 may be configured to deliver CRT, ATP therapy, bradycardia therapy, and/or other therapy types via leads 212 implanted within the heart. In some embodiments, MD 200 may additionally be configured to provide defibrillation/cardioversion therapy.

In some instances, MD 200 may be an implantable cardioverter-defibrillator (ICD). In such embodiments, MD 200 may include one or more leads implanted within a patient's heart. MD 200 may also be configured to sense electrical cardiac signals, determine occurrences of tachyarrhythmias based on the sensed electrical cardiac signals, and deliver defibrillation and/or cardioversion therapy in response to determining an occurrence of a tachyarrhythmia (for example by delivering defibrillation and/or cardioversion pulses to the heart of the patient). In other embodiments, MD 200 may be an SICD. In embodiments where MD 200 is an SICD, one of leads 212 may be a subcutaneously implanted lead. In at least some embodiments where MD 200 is an SICD, MD 200 may include only a single lead which is implanted subcutaneously but outside of the chest cavity, however this is not required.

In some embodiments, MD 200 may not be an implantable medical device. Rather, MD 200 may be a device external to the patient's body, and electrodes 214 may be skin-electrodes that are placed on a patient's body. In such embodiments, MD 200 may be able to sense surface electrical signals (e.g. electrical cardiac signals that are generated by the heart or electrical signals generated by a device implanted within a patient's body and conducted through the body to the skin). MD 200 may further be configured to deliver various types of electrical stimulation therapy, including, for example, defibrillation therapy via skin-electrodes 214.

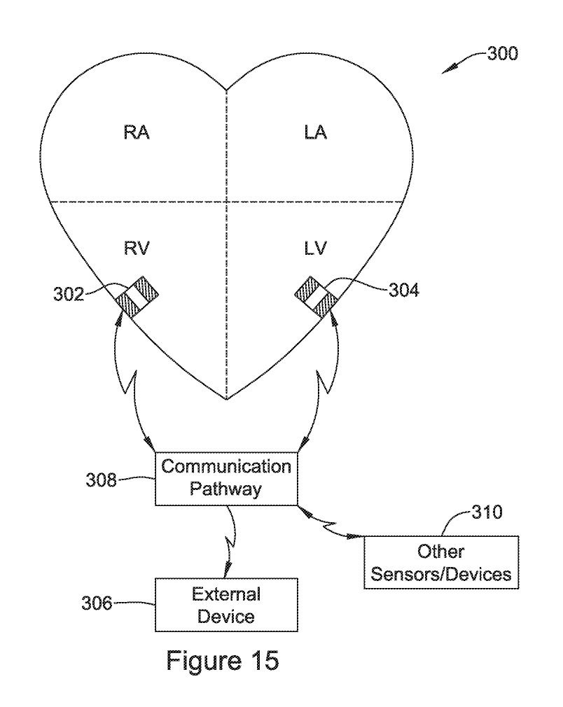

FIG. 15 illustrates an embodiment of a medical device system and a communication pathway through which multiple medical devices 302, 304, 306, and/or 310 of the medical device system may communicate. In the embodiment shown, medical device system 300 may include LCPs 302 and 304, external medical device 306, and other sensors/devices 310. External device 306 may be a device disposed external to a patient's body, as described previously with respect to MD 200. In at least some examples, external device 306 may represent an external support device such as a device programmer, as will be described in more detail below. Other sensors/devices 310 may be any of the devices described previously with respect to MD 200, such as ICPs, ICDs, and SICDs. Other sensors/devices 310 may also include various diagnostic sensors that gather information about the patient, such as accelerometers, blood pressure sensors, or the like. In some cases, other sensors/devices 310 may include an external programmer device that may be used to program one or more devices of system 300.

Various devices of system 300 may communicate via communication pathway 308. For example, LCPs 302 and/or 304 may sense intrinsic cardiac electrical signals and may communicate such signals to one or more other devices 302/304, 306, and 310 of system 300 via communication pathway 308. In one embodiment, one or more of devices 302/304 may receive such signals and, based on the received signals, determine an occurrence of an arrhythmia. In some cases, device or devices 302/304 may communicate such determinations to one or more other devices 306 and 310 of system 300. In some cases, one or more of devices 302/304, 306, and 310 of system 300 may take action based on the communicated determination of an arrhythmia, such as by delivering a suitable electrical stimulation to the heart of the patient. One or more of devices 302/304, 306, and 310 of system 300 may additionally communicate command or response messages via communication pathway 308.

The command messages may cause a receiving device to take a particular action whereas response messages may include requested information or a confirmation that a receiving device did, in fact, receive a communicated message or data.

It is contemplated that the various devices of system 300 may communicate via pathway 308 using RF signals, inductive coupling, optical signals, acoustic signals, or any other signals suitable for communication. Additionally, in at least some embodiments, the various devices of system 300 may communicate via pathway 308 using multiple signal types. For instance, other sensors/device 310 may communicate with external device 306 using a first signal type (e.g. RF communication) but communicate with LCPs 302/304 using a second signal type (e.g. conducted communication). Further, in some embodiments, communication between devices may be limited. For instance, as described above, in some embodiments, LCPs 302/304 may communicate with external device 306 only through other sensors/devices 310, where LCPs 302/304 send signals to other sensors/devices 310, and other sensors/devices 310 relay the received signals to external device 306.

In some cases, the various devices of system 300 may communicate via pathway 308 using conducted communication signals. Accordingly, devices of system 300 may have components that allow for such conducted communication. For instance, the devices of system 300 may be configured to transmit conducted communication signals (e.g. a voltage and/or current waveform punctuated with current and/or voltage pulses, referred herein as electrical communication pulses) into the patient's body via one or more electrodes of a transmitting device, and may receive the conducted communication signals via one or more electrodes of a receiving device. The patient's body may “conduct” the conducted communication signals from the one or more electrodes of the transmitting device to the electrodes of the receiving device in the system 300. In such embodiments, the delivered conducted communication signals may differ from pacing pulses, defibrillation and/or cardioversion pulses, or other electrical stimulation therapy signals. For example, the devices of system 300 may deliver electrical communication pulses at an amplitude/pulse width that is sub-threshold. That is, the communication pulses have an amplitude/pulse width designed to not capture the heart. In some cases, the amplitude/pulse width of the delivered electrical communication pulses may be above the capture threshold of the heart, but may be delivered during a refractory period of the heart and/or may be incorporated in or modulated onto a pacing pulse, if desired.

Additionally, unlike normal electrical stimulation therapy pulses, the electrical communication pulses may be delivered in specific sequences which convey information to receiving devices. For instance, delivered electrical communication pulses may be modulated in any suitable manner to encode communicated information. In some cases, the communication pulses may be pulse width modulated and/or amplitude modulated.

Alternatively, or in addition, the time between pulses may be modulated to encode desired information. In some cases, a predefined sequence of communication pulses may represent a corresponding symbol (e.g. a logic “1” symbol, a logic “0” symbol, an ATP therapy trigger symbol, etc.). In some cases, conducted communication pulses may be voltage pulses, current pulses, biphasic voltage pulses, biphasic current pulses, or any other suitable electrical pulse as desired.

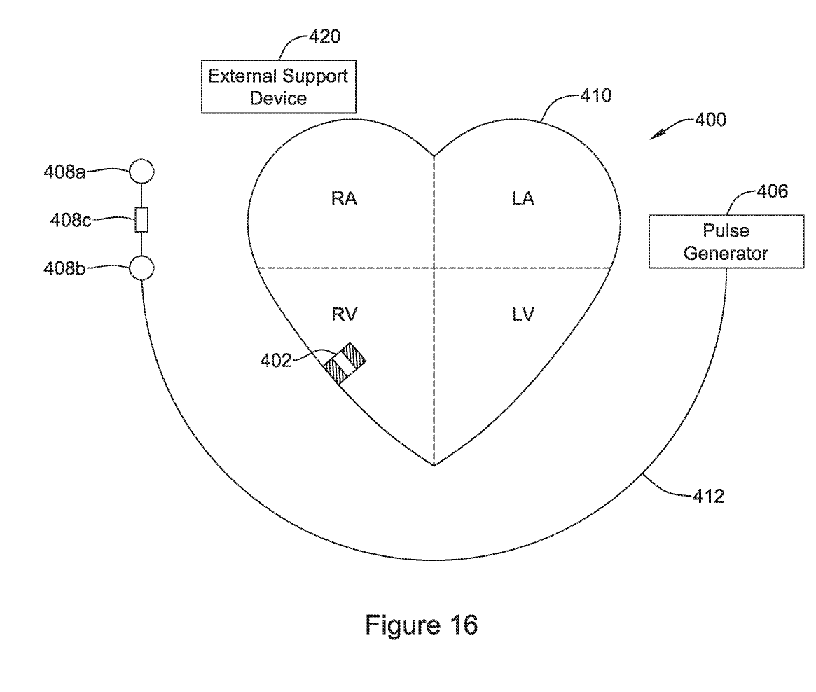

FIG. 16 depicts an illustrative medical device system 400 that may be configured to operate together. For example, system 400 may include multiple devices that are implanted within a patient and are configured to sense physiological signals, determine occurrences of cardiac arrhythmias, and deliver electrical stimulation to treat detected cardiac arrhythmias. In some embodiments, the devices of system 400 may be configured to determine occurrences of dislodgment of one or more devices of system 400. In FIG. 16, an LCP 402 is shown fixed to the interior of the right ventricle of the heart 410, and a pulse generator 406 is shown coupled to a lead 412 having one or more electrodes 408a-408c. In some cases, pulse generator 406 may be part of a subcutaneous implantable cardioverter-defibrillator (SICD), and the one or more electrodes 408a-408c may be positioned subcutaneously adjacent the heart. LCP 402 may communicate with the SICD, such as via communication pathway 308. The locations of LCP 402, pulse generator 406, lead 412, and electrodes 408a-c depicted in FIG. 16 are just exemplary. In other embodiments of system 400, LCP 402 may be positioned in the left ventricle, right atrium, or left atrium of the heart, as desired. In still other embodiments, LCP 402 may be implanted externally adjacent to heart 410 or even remote from heart 410.

Medical device system 400 may also include external support device 420. External support device 420 can be used to perform functions such as device identification, device programming and/or transfer of real-time and/or stored data between devices using one or more of the communication techniques described herein, or other functions involving communication with one or more devices of system 400. As one example, communication between external support device 420 and pulse generator 406 can be performed via a wireless mode, and communication between pulse generator 406 and LCP 402 can be performed via a conducted communication mode. In some embodiments, communication between LCP 402 and external support device 420 is accomplished by sending communication information through pulse generator 406. However, in other embodiments, communication between the LCP 402 and external support device 420 may be via a communication module.

FIG. 16 only illustrates one example embodiment of a medical device system that may be configured to operate according to techniques disclosed herein. Other example medical device systems may include additional or different medical devices and/or configurations. For instance, other medical device systems that are suitable to operate according to techniques disclosed herein may include additional LCPs implanted within the heart. Another example medical device system may include a plurality of LCPs with or without other devices such as pulse generator 406, with at least one LCP capable of delivering defibrillation therapy. Still another example may include one or more LCPs implanted along with a transvenous pacemaker and with or without an implanted SICD. In yet other embodiments, the configuration or placement of the medical devices, leads, and/or electrodes may be different from those depicted in FIG. 16. Accordingly, it should be recognized that numerous other medical device systems, different from system 400 depicted in FIG. 16, may be operated in accordance with techniques disclosed herein. As such, the embodiment shown in FIG. 16 should not be viewed as limiting in any way.

Those skilled in the art will recognize that the present disclosure may be manifested in a variety of forms other than the specific embodiments described and contemplated herein. For instance, as described herein, various embodiments include one or more modules described as performing various functions. However, other embodiments may include additional modules that split the described functions up over more modules than that described herein. Additionally, other embodiments may consolidate the described functions into fewer modules.

Although various features may have been described with respect to less than all embodiments, this disclosure contemplates that those features may be included on any embodiment. Further, although the embodiments described herein may have omitted some combinations of the various described features, this disclosure contemplates embodiments that include any combination of each described feature. Accordingly, departure in form and detail may be made without departing from the scope and spirit of the present disclosure as described in the appended claims.

We claim:

1. A leadless cardiac pacemaker (LCP) configured to sense and pace a patient's heart, the LCP comprising:
a housing;
two or more electrodes secured relative to the housing and configured to receive electrical signals emanating from the patient's heart;
a controller coupled to the two or more electrodes, the controller configured to receive electrical signals from the two or more electrodes indicative of a heartbeat of the patient's heart, the received electrical signals including one or more of a Q, an R, and an S feature, as well as a T feature of a QRST complex, the controller configured to determine a first measure of cardiac load based at least in part on a delay between the Q or R feature and the T feature for the heart beat;
an accelerometer configured to sense an activity level of the patient, the controller configured to determine a second measure of cardiac load based at least in part on the activity level of the patient;
the controller further configured to determine a pacing rate based at least in part upon the first measure of cardiac load and to pace the patient's heart in accordance with the determined pacing rate; and
the controller further configured to utilize the second measure of cardiac load to act as a governor to the determined pacing rate.

2. The LCP of claim 1, wherein the controller is configured to determine the first measure of cardiac load based at least in part on a delay between the R feature and the T feature for the heartbeat.

3. The LCP of claim 1, wherein the controller is configured to receive electrical signals from the two or more electrodes indicative of a plurality of heartbeats, and the controller is configured to determine the first measure of cardiac load based at least in part on a delay between the Q or R feature and the T feature of each of two or more heartbeats.

4. The LCP of claim 1, wherein the controller is configured to:
repeatedly activate and deactivate the accelerometer to intermittently sense the activity level of the patient; and
determine the second measure of cardiac load based at least in part on two or more intermittently sensed activity levels of the patient.

5. The LCP of claim 1, wherein the controller is configured to act as a governor by determining a pacing rate limit based at least in part on the second measure of cardiac load, and to limit the determined pacing rate based on the pacing rate limit.

6. The LCP of claim 5, wherein the pacing rate limit is a function of the second measure of cardiac load, and the function is not a constant function.

7. The LCP of claim 1, wherein the controller is configured to determine the pacing rate based at least in part upon the first measure of cardiac load using a linear function having a non-zero slope.

8. The LCP of claim 1, wherein the accelerometer is configured to sense non-cardiac movement of the patient, and the activity level of the patient is based on the sensed non-cardiac movement of the patient.

9. The LCP of claim 1, wherein the accelerometer is configured to sense cardiac movement, and the activity level of the patient is based on the sensed cardiac movement.

10. The LCP of claim 1, wherein the accelerometer is configured to sense non-cardiac movement of the patient and cardiac movement, and the activity level of the patient is based at least in part on the sensed non-cardiac movement of the patient and the sensed cardiac movement.

11. A leadless cardiac pacemaker (LCP) configured to sense and pace a patient's heart, the LCP comprising:
a housing;
two or more electrodes secured relative to the housing and configured to receive electrical signals emanating from the patient's heart;
a controller coupled to the two or more electrodes, the controller configured to receive electrical signals from the two or more electrodes and determine a first measure of cardiac load based on the received electrical signals;
a sensor configured to provide a second measure of cardiac load, the sensor operatively coupled to the controller;
the controller further configured to determine a pacing rate based at least in part upon the first measure of cardiac load and to pace the patient's heart in accordance with the determined pacing rate; and
the controller further configured to use the second measure of cardiac load to determine a pacing rate limit, and to limit the determined pacing rate based on the pacing rate limit.

12. The leadless cardiac pacemaker (LCP) of claim 11, wherein the first measure of cardiac load is representative of how quickly the patient's heart prepares for a subsequent heartbeat.

13. The leadless cardiac pacemaker (LCP) of claim 11, wherein the first measure of cardiac load is representative of a time required for ventricle depolarization and repolarization.

14. The leadless cardiac pacemaker (LCP) of claim 11, wherein the first measure of cardiac load comprises a RT interval.

15. The leadless cardiac pacemaker (LCP) of claim 11, wherein the second measure of cardiac load is representative of gross movement of the patient.

16. The leadless cardiac pacemaker (LCP) of claim 11, wherein the sensor comprises an accelerometer and the second measure of cardiac load is derived at least in part from a signal outputted from the accelerometer.

17. The leadless cardiac pacemaker (LCP) of claim 11, wherein the controller is further configured to temporarily ignore any changes in the first measure of cardiac load, for at least two heartbeats, in determining the pacing rate.

18. The leadless cardiac pacemaker (LCP) of claim 11, wherein the controller is further configured to determine a plurality of first measures of cardiac load over time based on the received electrical signals, and to determine the pacing rate based on a moving average of two or more of the plurality of first measures of cardiac load.

19. A leadless cardiac pacemaker (LCP) configured to sense and pace a patient's heart, the LCP comprising:
two or more electrodes configured to receive electrical signals emanating from the patient's heart;
a controller coupled to the two or more electrodes configured to ascertain a first measure of cardiac load based at least in part upon electrical signals received by the two or more electrodes;
the controller further configured to receive a second measure of cardiac load;
the controller further configured to determine a pacing rate based at least in part upon the first measure of cardiac load and the second measure of cardiac load; and
the controller further configured to pace the patient's heart in accordance with the determined pacing rate.

20. The leadless cardiac pacemaker (LCP) of claim 19, wherein the first measure of cardiac load comprises a measure that responds more quickly to changes in cardiac activity than the second measure of cardiac load.
